(19) United States
(12) Patent Application Publication
(10) Pub. No.: US 2006/0099702 A1
(43) Pub. Date: May 11, 2006

(54) Agent-containing Bacillus natto culture extract and preservation method of Bacillus natto culture extract

(75) Inventors: Tsuyoshi Sugiyama, Kanagawa (JP); Syogo Imanishi, Tokyo (JP); Natsuko Koga, Kanagawa (JP)

Correspondence Address: ARMSTRONG, KRATZ, QUINTOS, HANSON & BROOKS, LLP, 1725 K STREET, NW, SUITE 1000, WASHINGTON, DC, 20006, US

(73) Assignee: OTO CORPORATION, Odawara-shi (JP)

(21) Appl. No.: 11/266,183

(22) Filed: Nov. 4, 2005

(30) Foreign Application Priority Data
Nov. 5, 2004 (JP) 2004-322813
Oct. 7, 2005 (JP) 2005-295699

(51) Int. Cl. C12N 1/20

(52) U.S. Cl. 435/252.31

(57) ABSTRACT

The present invention provides an agent containing Bacillus natto culture extract capable of preventing the agent itself from becoming darker in color, natto-specific odor, and preventing degradation of active ingredients in the Bacillus natto culture extract such as natto-kinase. The present invention also provides effective preservation methods of the Bacillus natto culture extract.

To this end, the agent containing Bacillus natto culture extract of the present invention comprises a Bacillus natto culture extract, and any one of a reducing sugar and a reducing sugar derivative. The reducing sugar derivative is preferably the one having no reducing terminal. The invention is particularly effective when used in transparent capsules, because it has an effect of preventing itself from changing in color. Further, a Bacillus natto culture extract preserved such that the extract and any of the reducing sugar and the reducing sugar derivative are mixed therein enables preventing degeneration thereof.

### BACKGROUND OF THE INVENTION

1. Field of the Invention

The object of the present invention relates to an agent-containing Bacillus natto culture extract capable of preventing itself from becoming darker in color with the lapse of time, suppressing natto-specific odor, and inhibiting degradation of active ingredients therein and relates to the preservation method of the Bacillus natto culture extract.

2. Description of the Related Art

Bacillus natto culture extract contains active ingredients such as natto-kinase, and polyglutamic acid abundantly. It is clearly disclosed that in particular, natto-kinase has thrombolytic effect, thrombogenicity inhibitory effect, and blood flowability improving effect in Japanese Patent Application Laid-Open UP-A) No. 2001-352929 and on pp. 333-338, No. 4 (2002), Vol. 25 of Journal of Analytical Bio-science, published by The Society for Analytical Bio-Science, and natto-kinase is one of the ingredients getting so much attention in the field.

However, people who dislike natto hardly able to take natto so much, although they know that natto-kinase is an active ingredient for their health benefits. The reason why these people dislike natto is that natto has a specific odor and a specific stickiness.

With a background like that, Japanese Patent Application Laid-Open (JP-A) No. 11-18712 tried to prepare a capsulated agent containing Bacillus natto culture extract so that even people who do no like natto-specific odor and its stickiness can easily take Bacillus natto culture extracts such as natto-kinase.

However, when a Bacillus natto culture extract is produced into an agent, there is a problem that the agent becomes darker in color with the lapse of time. In addition there is also a problem that the natto-specific odor of the agent-containing Bacillus natto culture extract will become rancid as it will discolor with time. Since agents typified by capsulated agents are generally taken by mouth, an agent that had become darker in color and is giving off a strong odor does not make favorable impressions on consumers and brings suffering to the consumers when they take such an agent-containing Bacillus natto culture extract.

As a method for resolving the above problems, it is conceivable that the natto-specific odor and the discoloration of the agents can be prevented by adding absorbents and antiseptic agents. However, when absorbents and antiseptic agents are added to agents, a new problem arises. It means that various active ingredients in the Bacillus natto culture extract degrade.

### SUMMARY OF THE INVENTION

It is therefore an object of the present invention to provide an agent-containing Bacillus natto culture extract capable of inhibiting natto-specific odor and discoloration thereof and being hard to degrade active ingredients contained in the Bacillus natto culture extract such as natto-kinase.

Secondly, the object of the present invention is to provide a preservation method of the agent-containing Bacillus natto culture extract capable of inhibiting the natto-specific odor and discoloration thereof and being hard to degrade active ingredients contained in the Bacillus natto culture extract such as natto-kinase.

The agent-containing Bacillus natto culture extract of the present invention comprises a Bacillus natto culture extract and any one of a reducing sugar and a reducing sugar derivative.

The preservation method of the Bacillus natto culture extract of the present invention comprises preserving a Bacillus natto culture extract so that the Bacillus natto culture extract and any one of a reducing sugar and a reducing sugar derivative are mixed.

### BREIF DESCRIPTION OF THE DRAWINGS

The patent or application file contains at least one drawing executed in color. Copies of this patent or patent application publication with color drawings will be provided by the Office upon request and payment of the necessary fee.

FIG. 1 is a picture of a capsulated agent containing Bacillus natto culture extract in a state of its color being virtually unchanged.

FIG. 2 is a picture of a capsulated agent containing Bacillus natto culture extract in a state of its color being brownish in color.

FIG. 3 is a picture of a capsulated agent containing Bacillus natto culture extract in a state of its color being discolored into black.

### DESCRIPTION OF THE PREFERRED EMBODIMENTS

(Agent-Containing Bacillus Natto Culture Extract)

The agent-containing Bacillus natto culture extract of the present invention comprises a Bacillus natto culture extract and any one of a reducing sugar and a reducing sugar derivative and further comprises other additives in accordance with the necessity.

Hereinafter, the agent-containing Bacillus natto culture extract of the present invention will be described.

—Bacillus Natto Culture Extract—

The Bacillus natto culture extract can be obtained by isolating Bacillus natto from a culture which is obtained by culturing the Bacillus natto and subjecting it to other treatments in accordance with the necessity.

It is conceivable that useful substances such as polyglutamic acids and natto-kinase are contained in the Bacillus natto culture extract. A Bacillus natto culture extract containing natto-kinase has thrombolytic effect, thrombogenicity inhibitory effect, and blood flowability improving effect, therefore, the Bacillus natto culture extract is preferably a Bacillus natto culture extract containing natto kinase.

—Bacillus Natto —

Examples of the Bacillus natto include Bacillus subtilises, of which Bacillus subtilis natto is particularly preferable in consideration of safety and production quantity of natto-kinase.

The strain of the Bacillus natto is not particularly limited, may be suitably selected in accordance with the intended use, and examples thereof include commercially available Bacillus nattos such as Takahashi strain (produced by Yuzo Takahashi Laboratory in Yamagata), Naruse strain (produced by Naruse Fermentation Chemical Laboratory KK in Tokyo), Miyagino strain (produced by Miyagino Natto Lab Limited Company in Sendai), Asahi strain (produced by Asahi Kogyo KK in Tokyo), Nitto strain (produced by Nitto Yakuhin KK in Kyoto), and Meguiro strain (produced by Meguro Laboratory KK in Osaka), and Unnan SL-001 strain.

—Culture Medium —

A culture medium used for the culture of the Bacillus natto is not particularly limited, provided that the Bacillus natto to be cultured contains nutritional elements that can be used as nutrient sources, and examples of the culture medium include synthetic culture media, semisynthetic culture media, and natural culture media. It is possible to use the same culture media as conventional ones typically used for culturing a Bacillus natto.

The nutrient source used for the culture is not particularly limited, and may be suitably selected in accordance with the intended use. Examples thereof include bean curd refuses, soybeans, soybean pastes, soybean broths which are side products when producing natto, tofu cakes which are side products when producing tofu and deep-fried tofu, soybean cakes which are side products when producing oils derived from soybeans, seed coats of soybeans which are side products when producing soybean pastes, and various culture ingredients such as soy proteins. Each of these nutrient sources may be used alone or in combination with two or more.

Further, auxiliary ingredients other than the nutrient sources can also be added to the culture medium. Examples of the auxiliary ingredients include carbon sources, nitrogen sources, inorganic salts, and other nutritional elements which are needed for Bacillus natto to develop its life processes. Each of these auxiliary ingredients may be used alone or in combination with two or more.

The carbon sources are not particularly limited, provided that a strain to be used can grow sufficiently, and may be suitably selected in accordance with the intended use. Examples thereof include carbohydrates such as starches or compositional fractions thereof, roasted dextrins, processed starches, starch derivatives, physically processed starches, soluble starches, amyloses, amylopectins, maltoligosaccharides, cyclodextrins, pullulans, corn starches, potato starches, sweet potato starches, dextrins, glycerins, sorbitols, liquid malt, glucoses. Each of these carbon sources may be used alone or in combination with two or more.

Among the carbon sources, one or more selected from glucoses or starches are preferably used to increase the production quantity of natto kinase.

The nitrogen sources are not particularly limited, provided that a strain to be used can grow sufficiently, and may be suitably selected in accordance with the intended use. Examples thereof include organic nitrogen compounds, inorganic nitrogen compounds, and mixtures thereof.

The organic nitrogen compounds are not particularly limited, and may be suitably selected in accordance with the intended use. Examples thereof include meat extracts, malt extracts, peptones, polypeptones derived from soybeans such as polypeptone-S, yeast extracts, flavor liquid or acid hydrolysates of soybean proteins, soybean powders, milk caseins, casamino acids, various amino acids, and corn steep liquors. Each of these organic nitrogen compounds may be used alone or in combination with two or more.

The inorganic nitrogen compounds are not particularly limited, and may be suitably selected in accordance with the intended use. Examples thereof include ammonias, ammonium sulfates, ammonium salts of ammonium sulfates and ammonium chlorides, nitrates such as sodium nitrates, and ureas. Each of these inorganic nitrogen compounds may be used alone or in combination with two or more.

Among these nitrogen sources, in order to increase the production quantity of natto-kinase, it is preferred to add one or more selected from any one of polypeptones derived from soybeans such as polypeptone-S and soybean powders to the culture medium.

The inorganic salts are not particularly limited, provided that a strain to be used can grow sufficiently, and may be suitably selected in accordance with the intended use. Examples thereof include phosphates of magnesium, manganese, calcium, sodium, potassium, copper, iron, and zinc; hydrochloride salts, sulfate salts, and acetate salts. Each of these inorganic salts may be used alone or in combination with two or more.

For the form of the culture medium containing the above-mentioned nutrition sources, it may be formed in solid form or in liquid form, however, a liquid culture medium is preferably used because of its excellence in productivity.

The culture method is not particularly limited, may be suitably selected in accordance with the intended use. Examples thereof include static culture, shaking culture, and agitation culture, of which shaking culture is preferable when a liquid culture medium is used.

Culture conditions of the Bacillus natto are not particularly limited, may be suitably selected in accordance with the strain to be used, the composition of the culture medium, and the culture method. The conditions under which the strain to be used can grow are preferably employed.

The culture temperature is typically 20° C. to 45° C., and preferably 37° C. to 40° C. The suitable pH of the culture medium is typically 6.0 to 9.5, and more preferably 7.0 to 8.5.

The amount of the Bacillus natto to be inoculated is preferably 2% by mass to 10% by mass to the amount of the culture medium.

The number of days for the culture period is preferably 2 days to 7 days.

—Isolation of Bacillus Natto—

The isolation method of the Bacillus natto is not particularly limited, and examples thereof include filtrations, ultrafiltrations, and centrifugations. Each of these methods may be used alone or in combination with two or more. The solids or the like that had been added to a culture medium can be removed together with the Bacillus natto from the culture medium in which the Bacillus natto had been cultured by means of these methods.

—Other Treatments —

Other treatments are not particularly limited, and examples thereof include concentration, drying, and purification.

The concentration method is not particularly limited, and examples thereof include concentration under reduced pressure, and concentration by heating, of which the concentration under reduced pressure is preferably used because proteins, enzymes, or the like are susceptible to thermal denaturation.

The drying method is not particularly limited, and examples thereof include freeze-drying, air-drying, and vacuum heat-drying. In addition, a Bacillus natto culture extract in powder form can be obtained by crushing a Bacillus natto culture extract which has been dried by means of any of the drying methods.

Doing the purification is advantageous in that it is possible to obtain a Bacillus natto culture extract containing a high concentration of natto-kinase and a Bacillus natto culture extract in which most of natto-kinase is removed.

For the purification method, those known in the art can be used. For example, the following purification methods can be employed.

A powdered Bacillus natto culture extract is dissolved in a Tris-HCl buffer solution, and the solution is fractioned using a 25% by mass ammonium sulfate and then centrifuged to filter and remove the sediment. Thereafter, to the filtrate, an ion-exchange resin was poured and stirred to remove the supernatant liquid. The concentration of the natto kinase can be improved by 200 times to 1,000 times by adding the Tris-HCl buffer solution to the ion-exchange resin to elute the natto-kinase.

Conversely, it is also possible to obtain a Bacillus natto culture extract in which most of natto-kinase is removed by the following method. The powdered Bacillus natto culture extract is dissolved in a Tris-HCl buffer solution, and the solution is fractioned using a 25% by mass ammonium sulfate and then centrifuged to filter and remove the sediment. Thereafter, to the filtrate, an ion-exchange resin was poured and stirred to make the natto-kinase absorbed in the ion-exchange resin, and then the supernatant liquid is concentrated to thereby obtain a Bacillus natto culture extract with most of natto-kinase being removed.

The used form of the Bacillus natto culture extract after subjecting to the purification is not particularly limited, for example, the Bacillus natto culture extract in liquid form and in paste form or powder form obtained and treated by means of the concentration and the drying may be used.

—Reducing Sugar—

The reducing sugar is not particularly limited, may be suitably selected in accordance with the intended use, and examples thereof include glucose, fructose, and maltose. Each of these reducing sugars may be used alone or in combination with two or more. Among these reducing sugars, maltose is preferably used from the perspective of its excellence in preventing the agent from becoming darker in color, preventing degradation of active ingredients therein, and suppressing natto-specific odor.

—Reducing Sugar Derivative—

The reducing sugar derivative is not particularly limited, may be suitably selected in accordance with the intended use, and examples thereof include reducing sugar derivatives that the reducing terminal (aldehyde group) is hydrogenated, reducing sugar derivatives that the reducing terminal is oxidized, reduced dextrin obtained by hydrogenating maltodextrin, and reduced dextrin obtained by acid-hydrolyzing a starch emulsion and then hydrolyzing the acid hydrolyzed starch emulsion in a range from DE15 to DE22 through the use of α-amylase followed by hydrogenation. Each of these reducing sugar derivatives may be used alone or in combination with two or more. Among these reducing sugar derivatives, reduced dextrin is preferably used from the perspective of its excellence in preventing the agent from becoming darker in color, preventing degradation of active ingredients therein, and suppressing natto-specific odor.

Examples of the hydrogenated reducing sugar derivatives include sugar alcohols. The sugar alcohols are not particularly limited, may be suitably selected in accordance with the intended use, and examples thereof include sorbitol, maltitol, erythritol, xylitol, and lactitol. Each of these sugar alcohols may be used alone or in combination with two or more.

Examples of the oxidized reducing sugar derivatives include aldonic acids with only the reducing terminal being oxidized such as gluconic acids, and aldaric acids with both the terminals being oxidized, not only the reducing terminal, such as glucaric acids. Each of these oxidized reducing sugar derivatives may be used alone or in combination with two or more.

The reducing sugar derivatives having no reducing terminal such as the sugar alcohols, the aldonic acids, and the aldaric acids are preferably used, because they are hardly likely to do Maillard reaction with amino acids included in the Bacillus natto culture extract as well as with amino groups contained in peptide.

Among the reducing sugar derivatives having no reducing terminal, sugar alcohols are preferably used, because aldonic acids and aldaric acids acidify the Bacillus natto culture extract.

The reducing sugars and the reducing sugar derivatives may be used alone or in combination with two or more. It is also possible to use mixtures of the reducing sugars and the reducing sugar derivatives.

—Other Additives

The additives are not particularly limited, may be suitably selected in accordance with the intended use, and examples thereof include vehicles such as sucrose, mannite, corn starch, synthetic gums or natural gums, and crystalline celluloses; bonding agents such as starches, cellulose derivatives, gum arabic, gelatins, polyvinylpyrolidone, disintegrating agents such as calcium carboxymethyl cellulose, sodium carboxymethyl cellulose, starches, corn starch, and alginate sodium; gloss agents such as talcs, magnesium stearates, and sodium stearates; fillers such as calcium carbonates, sodium carbonates, calcium phosphates, and sodium phosphates; diluents, and various vitamins.

The added amount of the reducing sugars and the reducing sugar derivatives to the Bacillus natto culture extract is preferably 50 parts by mass to 150 parts by mass, and more preferably 80 parts by mass to 120 parts by mass relative to 100 parts by mass of solids of the Bacillus natto culture extract.

When the added amount of the reducing sugars and the reducing sugar derivatives relative to 100 parts by mass of solids of the Bacillus natto culture extract is less than 50 parts by mass, it is likely to have less inhibitive effect of preventing the agent containing the Bacillus natto culture extract from becoming darker in color, and when the added amount of the reducing sugars and the reducing sugar derivatives is more than 150 parts by mass, the content of the Bacillus natto culture extract in the agent containing the Bacillus natto culture extract is reduced.

The form of the agent containing the Bacillus natto culture extract is not particularly limited, may be suitably selected in accordance with the intended use, and examples thereof include tablets, pills, powders, dusting powders, granules, syrups, solutions, suspensions, emulsions, and capsules.

Among these forms, the capsules are preferably used because it is possible to formulate not only Bacillus natto culture extracts in powder form and in granulated form but also in liquid form and in paste form. The capsules to be used for the capsulated agent may be soft capsules or hard capsules.

The capsules to be used for the capsulated agent may be transparent ones or opaque ones, however, the agent containing the Bacillus natto culture extract of the present invention can exert its effect more effectively when used in transparent capsules because it can prevent it from becoming darker in color.

The tablet agents or the like may be subjected to a sugar coating, a gelatin coating, an enteric coating, and a film coating, or the like through the use of a proper base for coating in accordance with the necessity.

The agent containing the Bacillus natto culture extract can be used as pharmaceuticals and health food products.

(Preservation Method of Bacillus Natto Culture Extract)

According to the preservation method of Bacillus natto culture extract of the present invention, a Bacillus natto culture extract is preserved such that the Bacillus natto culture extract and any one of the reducing sugar and the reducing sugar derivative are mixed.

Examples of the mixing method include agitation and mixing method, natural mixing method, kneading and mixing method, and mixing method using a ball mill, and the like.

The mixing temperature is preferably 15° C. to 60° C.

The mixed condition of the Bacillus natto and any one of the reducing sugar and the reducing sugar derivative is not particularly limited, may be suitably selected in accordance with the intended use, and examples thereof include mixed conditions where the reducing sugar and the reducing sugar derivative are dissolved in the Bacillus natto culture extract formed in a fluid form such as in liquid form or in paste form; mixed conditions where the Bacillus natto culture extract, the reducing sugar, and the reducing sugar derivative are mixed in a same granulated powder; mixed conditions where a granulated powder containing the Bacillus natto culture extract and a granulated powder containing any one of the reducing sugar and the reducing sugar derivative are mixed; mixed conditions where the Bacillus natto culture extract in powder form and any one of the reducing sugar in powder form and the reducing sugar derivative in powder form are mixed; and mixed conditions where the Bacillus natto culture extract and any one of the reducing sugar and the reducing sugar derivative are mixed in a same capsule.

For the mixed condition of the Bacillus natto and any one of the reducing sugar and the reducing sugar derivative, it is preferred that they are hermetically sealed for preservation. In the present invention, the sealed conditions mean not only sealing them into containers but also sealing them into capsules and into coatings.

The preservation method of the Bacillus natto culture extract is usable when providing agents and health food products in which the Bacillus natto culture extracts are used.

The agent containing the Bacillus natto culture extract can be used as pharmaceuticals and health food products. In addition, the preservation method of the Bacillus natto culture extract of the present invention can be used when producing pharmaceuticals and health food products in which the Bacillus natto culture extract is used.

According to the present invention, it is possible to present an agent containing Bacillus natto culture extract capable of solving various conventional problems stated above, preventing itself from changing in color (from becoming darker in color) with the lapse of time, suppressing the odor, and inhibiting the degradation of active ingredients contained in the Bacillus natto culture extract such as natto-kinase as well as to provide the preservation method of Bacillus natto culture extract.

### EXAMPLES

(Bacillus Natto Culture Extract)

[Culture of Bacillus Natto]

For the Bacillus natto, Takahashi strain of Bacillus natto was used in the present invention. Pure water was added to the Bacillus natto and then stirred so as to be in a suspension state.

Next, a standard agar culture medium (manufactured by NISSUI) was used to prepare a plate culture medium in a Schale. A small amount of the Bacillus natto was inoculated with the plate culture medium, and cultured at 37° C. for 24 hours to obtain a colony of the Bacillus natto on the plate culture medium.

[Preparation of Medium]

In a 500 ml conical flask, 1 g of glucose being a raw material, 4 g of powdery soybean protein (Solpy N.Y., manufactured by Nisshin Cosmo Foods Co., Ltd.), and 200 ml of a liquid culture medium comprising distilled water were poured and prepared, and the flask was held at a temperature of 120° C. for 30 minutes using an autoclave to sterilize the materials in the conical flask.

[Culture]

From the colony of the Bacillus natto which was cultured on the plate medium, the Bacillus natto was taken once using a platinum loop ear having a diameter of the platinum wire loop of 2 mm and inoculated to the liquid culture medium.

Next, the conical flask in which the liquid culture medium inoculated with the Bacillus natto was housed was shaken and circumvolved with maintaining the temperature of 40° C. without sealing the conical flask for 4 days to thereby culture the Bacillus natto.

[Isolation of Bacillus Natto]

After the Bacillus natto and impurities were removed from the obtained Bacillus natto culture solution through the use of a centrifugal separator, 0.2 moles of ammonium sulfate was added to the Bacillus natto culture solution, and then the solution was filtered through a glass fiber filter paper to thereby obtain a Bacillus natto culture extract as a filtrate.

[Concentration]

The Bacillus natto culture extract was concentrated under reduced pressure to obtain a 20% by weight of Bacillus natto culture extract in paste form.

[Reducing Sugar and Reducing Sugar Derivative]

Reduced dextrin (H-PDX, manufactured by Matsutani Chemical Industry Co., Ltd.) and sorbitol were used as the reducing sugar derivative, and a maltose (Amalty, manufactured by Towa Kasei Co., Ltd.) and fructose were used as the reducing sugar.

[Mixing]

The Bacillus natto culture extract in paste form and the reducing sugar or the reducing sugar derivative were kneaded and mixed.

[Drying]

The Bacillus natto culture extract containing the reducing sugar or the reducing sugar derivative was freeze-dried and crushed to thereby obtain a Bacillus natto culture extract containing the reducing sugar in powder form.

[Capsulation]

The Bacillus natto culture extract containing the reducing sugar or the reducing sugar derivative and the Bacillus natto culture extract containing the powdery reducing sugar or the powdery reducing sugar derivative were made to be included in soft capsules made from gelatin, glycerin ester, and beeswax to thereby obtain a capsulated agent.

<Evaluation of Change in Color into Black>

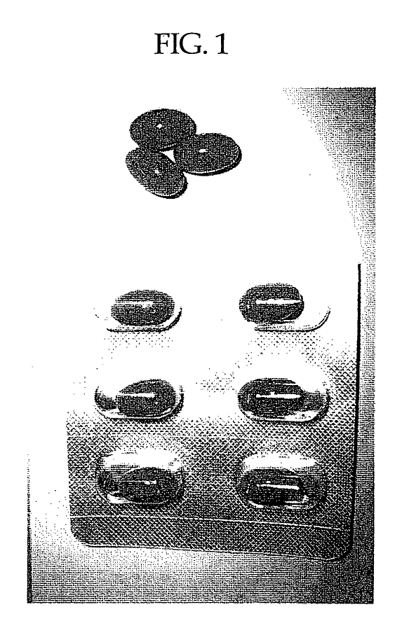

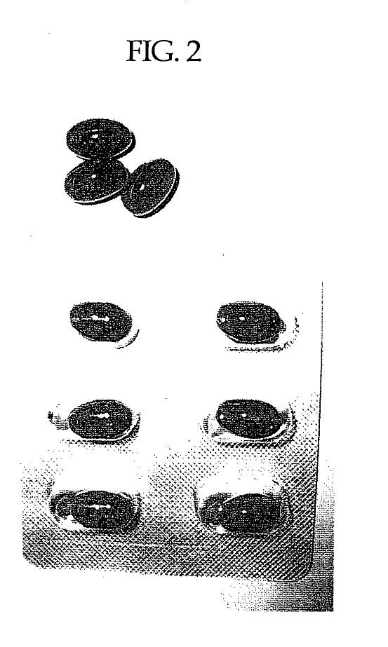

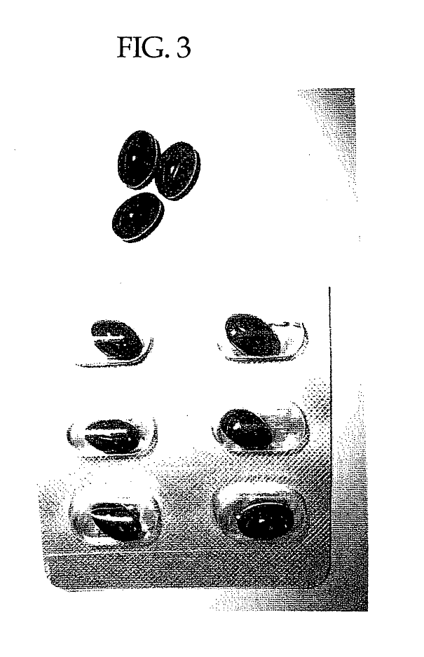

Each of these samples of the Bacillus natto culture extract were stored in a temperature-controlled room at both temperatures of 40° C. and 50° C. for 26 days, the change in color with time of each of these samples were visually observed four times in total one day later, 8 days later, 11 days later, and 26 days later of start of the preservation and evaluated in accordance with the following evaluation criteria. FIG. 1 shows a picture of a capsulated agent containing Bacillus natto culture extract in a state of its color being virtually unchanged. FIG. 2 shows a picture of a capsulated agent containing Bacillus natto culture extract in a state of its color being brownish in color, and FIG. 3 shows a picture of a capsulated agent containing Bacillus natto culture extract in a state of its color being discolored into black.

[Evaluation Criteria]

A: The agent in a state where the color was virtually unchanged was evaluated as A.

B: The agent in a state where the color was changed to brownish color in some degree was evaluated as B.

C: The agent in a state where the color was discolored into black was evaluated as C.

<Evaluation of Odor>

We inventors had two test subjects smell each of these samples to evaluate them in accordance with the following criteria.

[Evaluation Criteria]

A: The agent in a state where the sample did not give off a natto-specific odor was evaluated as A.

B: The agent in a state where the sample slightly gave off a natto-specific odor was evaluated as B.

C: The agent in a state where the sample definitely gave off a natto-specific odor was evaluated as C.

### Example 1

A Bacillus natto culture extract in paste form containing natto-kinase and a maltose (Amalty, manufactured by Towa Kasei Co., Ltd.) were mixed to form a paste. The added amount of the maltose to the Bacillus natto culture extract was 100 parts by mass relative to 100 parts by mass of solids of the Bacillus natto culture extract. The obtained paste was made to be included in soft capsules comprising gelatin, glycerin ester, and beeswax using a rotary method to obtain a capsulated agent.

We inventors had the two test subjects smell the capsulated agent, and it hardly gave off a natto-specific odor.

The capsulated agent was left in a temperature-controlled room at both temperatures of 40° C. and 50° C. and observed one day later, 8 days later, 11 days later, and 26 days later of start of the preservation, and no change in color was observed.

### Example 2

A Bacillus natto culture extract in paste form containing natto-kinase and a reduced dextrin (H-PDX, manufactured by Matsutani Chemical Industry Co., Ltd.) were mixed and freeze-dried to form a powder. The added amount of the reduced dextrin to the Bacillus natto culture extract was 100 parts by mass relative to 100 parts by mass of solids of the Bacillus natto culture extract.

The obtained powder was made to be included in soft capsules comprising gelatin, glycerin ester, and beeswax using a rotary method to obtain a capsulated agent.

We inventors had the two test subjects smell the capsulated agent, and it hardly gave off a natto-specific odor.

The capsulated agent was left in a temperature-controlled room at both temperatures of 40° C. and 50° C. and observed one day later, 8 days later, 11 days later, and 26 days later of start of the preservation, and no change in color was observed.

### Example 3

A Bacillus natto culture extract in paste form containing natto-kinase and a reduced dextrin (H-PDX, manufactured by Matsutani Chemical Industry Co., Ltd.) were mixed and freeze-dried to form a powder. The added amount of the reduced dextrin to the Bacillus natto culture extract was 100 parts by mass relative to 100 parts by mass of solids of the Bacillus natto culture extract.

The obtained powder and a crystalline cellulose powder (vehicle) were mixed to obtain a tablet agent of a Bacillus natto culture extract containing natto-kinase by means of the tablet press method.

We inventors had the two test subjects smell the tablet agent, and it hardly gave off a natto-specific odor.

The tablet agent was left in a temperature-controlled room at both temperatures of 40° C. and 50° C. and observed one day later, 8 days later, 11 days later, and 26 days later of start of the preservation, and no change in color was observed.

### Example 4

The paste obtained in Example 1 was diluted 20-fold to form a liquid agent of a Bacillus natto culture extract containing natto-kinase. The liquid agent was poured into a bottle, and the bottle was hermetically sealed.

We inventors had the two test subjects smell the liquid agent, and it hardly gave off a natto-specific odor.

The liquid agent was left in a temperature-controlled room at both temperatures of 40° C. and 50° C. and observed one day later, 8 days later, 11 days later, and 26 days later of start of the preservation, and no change in color was observed.

### Example 5

A Bacillus natto culture extract in paste form containing natto-kinase and a sorbitol were mixed to form a paste. The added amount of the sorbitol to the Bacillus natto culture extract was 100 parts by mass relative to 100 parts by mass of solids of the Bacillus natto culture extract. The obtained paste was made to be included in soft capsules comprising gelatin, glycerin ester, and beeswax using a rotary method to obtain a capsulated agent.

We inventors had the two test subjects smell the capsulated agent, and it hardly gave off a natto-specific odor.

The capsulated agent was left in a temperature-controlled room at both temperatures of 40° C. and 50° C. and observed one day later, 8 days later, 11 days later, and 26 days later of start of the preservation, and slight changes in color of the capsulated agent were observed at 40° C. at the 8 days later observation or later. Under the condition of 50° C., the capsulated agent slightly changed in color after the one later observation.

### Example 6

A capsulated agent containing Bacillus natto culture extract was obtained in the same manner as Example 1 except that 100 parts by mass of a fructose were used instead of the maltose.

We inventors had the two test subjects smell the capsulated agent, and it slightly gave off a natto-specific odor.

The capsulated agent was left in a temperature-controlled room at both temperatures of 40° C. and 50° C. and observed one day later, 8 days later, 11 days later, and 26 days later of start of the preservation, and the capsulated agent was changed in color after the 26 days later observation at both temperatures of 40° C. and 50° C.

### Example 7

A capsulated agent containing Bacillus natto culture extract was obtained in the same manner as Example 1 except that 40 parts by mass of the maltose (Amalty, manufactured by Towa Kasei Co., Ltd.) were used relative to 100 parts by mass of solids of the Bacillus natto culture extract.

We inventors had the two test subjects smell the capsulated agent, and it hardly gave off a natto-specific odor.

The capsulated agent was left in a temperature-controlled room at both temperatures of 40° C. and 50° C. and observed one day later, 8 days later, 11 days later, and 26 days later of start of the preservation, and a slight change in color was observed after the 26 days later observation at both temperatures of 40° C. and 50° C.

### Example 8

A capsulated agent containing Bacillus natto culture extract was obtained in the same manner as Example 1 except that 160 parts by mass of the maltose (Amalty, manufactured by Towa Kasei Co., Ltd.) were used relative to 100 parts by mass of solids of the Bacillus natto culture extract.

We inventors had the two test subjects smell the capsulated agent, and it hardly gave off a natto-specific odor.

The capsulated agent was left in a temperature-controlled room at both temperatures of 40° C. and 50° C. and observed one day later, 8 days later, 11 days later, and 26 days later of start of the preservation, and no change in color was observed.

### Example 9

A capsulated agent containing Bacillus natto culture extract was obtained in the same manner as Example 1 except that 40 parts by mass of a reduced dextrin (H-PDX, manufactured by Matsutani Chemical Industry Co., Ltd.) were used instead of the maltose.

We inventors had the two test subjects smell the capsulated agent, and it hardly gave off a natto-specific odor.

The capsulated agent was left in a temperature-controlled room at both temperatures of 40° C. and 50° C. and observed one day later, 8 days later, 11 days later, and 26 days later of start of the preservation, and a slight change in color was observed after the 26 days later observation at both temperatures of 40° C. and 50° C.

### Example 10

A capsulated agent containing Bacillus natto culture extract was obtained in the same manner as Example 1 except that 160 parts by mass of a reduced dextrin (H-PDX, manufactured by Matsutani Chemical Industry Co., Ltd.) were used instead of the maltose.

We inventors had the two test subjects smell the capsulated agent, and it hardly gave off a natto-specific odor.

The capsulated agent was left in a temperature-controlled room at both temperatures of 40° C. and 50° C. and observed one day later, 8 days later, 11 days later, and 26 days later of start of the preservation, and no change in color was observed.

### Comparative Example 1

A Bacillus natto culture extract in paste form containing natto-kinase and a powder starch were mixed to form a paste. The added amount of the powder starch to the Bacillus natto culture extract was 50 parts by mass relative to 100 parts by mass of solids of the Bacillus natto culture extract. The obtained paste was made to be included in soft capsules comprising gelatin, glycerin ester, and beeswax using a rotary method to obtain a capsulated agent.

We inventors had the two test subjects smell the capsulated agent, and it gave off a thick odor of natto.

The capsulated agent was left in a temperature-controlled room at both temperatures of 40° C. and 50° C. and observed one day later, 8 days later, 11 days later, and 26 days later of start of the preservation. The capsulated agent showed no change in color till the one day later observation, however, thereafter it gradually discolored from brownish yellow through brown to black with the time.

### Comparative Example 2

A Bacillus natto culture extract in paste form containing natto-kinase was made to be included in soft capsules comprising gelatin, glycerin ester, and beeswax to obtain a capsulated agent.

We inventors had the two test subjects smell the capsulated agent, and it gave off a thick odor of natto.

The capsulated agent was left in a temperature-controlled room at both temperatures of 40° C. and 50° C. and observed one day later, 8 days later, 11 days later, and 26 days later of start of the preservation. The capsulated agent showed no change in color till the one day later observation, however, thereafter it gradually discolored from brownish yellow through brown to black with the time.

### Comparative Example 3

A mixture of a Bacillus natto culture extract in paste form containing natto-kinase and a starch was diluted with 5 volume water to form a liquid agent. The added amount of the starch to the Bacillus natto culture extract was 100 parts by mass relative to 100 parts by mass of solids of the Bacillus natto culture extract. The liquid agent was poured into a bottle, and the bottle was hermetically sealed.

We inventors had the two test subjects smell the liquid agent, and it gave off a thick odor of natto.

The capsulated agent was left in a temperature-controlled room at both temperatures of 40° C. and 50° C. and observed one day later, 8 days later, 11 days later, and 26 days later of start of the preservation. The capsulated agent showed changes in color after the one day later observation.

### Comparative Example 4

A Bacillus natto culture extract in paste form containing natto-kinase was transferred into a bottle, and the bottle was hermetically sealed.

We inventors had the two test subjects smell the liquid agent, and it gave off a thick odor of natto.

The capsulated agent was left in a temperature-controlled room at both temperatures of 40° C. and 50° C. and observed one day later, 8 days later, 11 days later, and 26 days later of start of the preservation. Changes in color were observed after the one day later observation.

TABLE 1

| | | Ex. 1 | Ex. 2 | Ex. 3 | Ex. 4 | Ex. 5 | Ex. 6 | Ex. 7 | Ex. 8 | Ex. 9 | Ex. 10 |
|---|---|---|---|---|---|---|---|---|---|---|---|
| Bacillus natto culture extract | | 100 | 100 | 100 | 100 | 100 | 100 | 100 | 100 | 100 | 100 |
| Saccharides | Maltose | 100 | — | — | — | — | — | 40 | 160 | — | — |
| | Fructose | — | — | — | — | — | 100 | — | — | — | — |
| | Reduced | — | 100 | 100 | 100 | — | — | — | — | 40 | 160 |
| | dextrin | | | | | | | | | | |
| | Sorbitol | — | — | — | — | 100 | — | — | — | — | — |
| | Starch | — | — | — | — | — | — | — | — | — | — |
| Test of | One day later | A | A | A | A | A | A | A | A | A | A |
| discoloration | 8 days later | A | A | A | A | B | A | A | A | A | A |
| into black | 11 days later | A | A | A | A | B | A | A | A | A | A |
| (40° C.) | 26 days later | A | A | A | A | B | B | B | A | B | A |
| Test of | One day later | A | A | A | A | B | A | A | A | A | A |
| discoloration | 8 days later | A | A | A | A | B | A | A | A | A | A |
| into black | 11 days later | A | A | A | A | B | A | A | A | A | A |
| (40° C.) | 26 days later | A | A | A | A | B | B | B | A | B | A |

| | | | | | | | | | | |
|---|---|---|---|---|---|---|---|---|---|---|
| Odor Test | A | A | A | A | A | B | A | A | A | A |

TABLE 2

| | | Compara. | Compara. | Compara. | Compara. |
|---|---|---|---|---|---|
| | | Ex. 1 | Ex. 2 | Ex. 3 | Ex. 4 |
| Bacillus natto | | 100 | 100 | 100 | 100 |
| culture extract | | | | | |
| Saccha- | Maltose | — | — | — | — |
| rides | Reduced | — | — | — | — |
| | dextrin | | | | |
| | Sorbitol | — | — | — | — |
| | Starch | 100 | — | 100 | — |
| Test of | One day later | C | C | C | C |
| change | 8 days later | C | C | C | C |
| in color | 11 days later | C | C | C | C |
| into | 26 days later | C | C | C | C |
| black | | | | | |
| (40° C.) | | | | | |
| Test of | One day later | C | C | C | C |
| change | 8 days later | C | C | C | C |
| in color | 11 days later | C | C | C | C |
| into | 26 days later | C | C | C | C |
| black | | | | | |
| (50° C.) | | | | | |

| | | | | |
|---|---|---|---|---|
| Odor Test | C | C | C | C |

### Example 11

A Bacillus natto culture extract in paste form containing natto-kinase and a maltose (Amalty, manufactured by Towa Kasei Co., Ltd.) were mixed to prepare an agent containing Bacillus natto culture extract. The added amount of the maltose was 10% by mass relative to the total amount of solids of the Bacillus natto culture extract in paste form and the maltose.

The obtained agent containing the Bacillus natto culture extract was stored in a temperature-controlled room at a temperature of 40° C. and measured four times in total as to the activity of natto-kinase on the day of starting the preservation, 6 months later, 12 months later, 24 months layer. Table 3 shows the resultant activity degrees of the four times measurements.

(Measurement of Activity of Natto-Kinase)

—Preparation of Natto-Kinase—

The agent containing Bacillus natto culture extract prepared in Example 11 was diluted with a diluent which was prepared by the following Preparation 1 so that the difference in absorbance between a sample and a blank used in the measurement of the activity of natto-kinase (the difference between the degree of absorbance of a sample and the degree of absorbance of a blank) was 0.04 to 0.08, to thereby prepare a sample solution. It should be noted that the concentration of the sample solution having the difference in absorbance of 0.04 to 0.08 is 0.67 FU/g to 1.33 FU/g. Here, 1 FU is equivalent to approx. 50 IU of urokinase.

—Measurement of Activity—

In a test tube having a diameter of 15 mm and a length of 150 mm, 1.4 mL of a borax buffer solution which was prepared by the following Preparation 2 and 0.4 mL of a fibrinogen solution which was prepared by the following Preparation 3 were poured, and the test tube was heated in a temperature-controlled water bath at 37° C. for 5 minutes. Thereafter, 0.1 mL of a thrombin solution which was prepared by the following Preparation 4 was added in the test tube and stirred. The obtained solution was left at a temperature of 37° C. for 10 minutes, and then 0.1 mL of the sample solution was added, stirred for 5 seconds, and left at 37° C. Then, 20 minutes later and 40 minutes later from addition of the sample solution, the solution was stirredrespectively. Further, 60 minutes later, 2 mL of a 0.2 mol/L trichloroacetate solution which was prepared by the following Preparation 5 was added to the solution, stirred, and then left at 37° C. for 20 minutes. The obtained solution was poured in a microtest tube and centrifuged at 15,000×g for 5 minutes. The obtained supernatant was collected in an amount of 1 mL using a Pasteur pipette, and the absorbance (A_{T}) of the supernatant was measured at a wavelength of 275 nm.

In addition, in another test tube having a diameter of 15 mm and a length of 150 mm, 1.4 mL of a borax buffer solution which was prepared by the following Preparation 2 and 0.4 mL of a fibrinogen solution which was prepared by the following Preparation 3 were poured, and the test tube was heated in a temperature-controlled water bath at 37° C. for 5 minutes. Thereafter, 0.1 mL of a thrombin solution which was prepared by the following Preparation 4 was added in the test tube and stirred. The obtained solution was left in the temperature-controlled water bath at a temperature of 37° C. for 10 minutes, and then 2 mL of a 0.2 mL/L trichloroacetate solution was added to the solution, and stirred. Next, 0.1 mL of the sample solution was further added to the solution and stirred. This solution was left at 37° C. for 20 minutes. The obtained solution was poured in a microtest tube and centrifuged at 15,000×g for 5 minutes. The obtained supernatant was collected in an amount of 1 mL using a Pasteur pipette, and the absorbance (A_{B}) of the supernatant was measured at a wavelength of 275 nm.

Based on the measured degrees of absorbance (A_{T} and A_{B}), the activity of natto-kinase was measured using the following Equation 1.
Activity of Natto-Kinase (FU/g)=(A_{T}−A_{B})/0.01×1/60×1/0.1×D Equation 1

However, in Equation 1, D represents a dilution multiple of a sample.

—Preparation 1 (Preparation of Diluent)—

In a vessel, water was added to 0.344 g of calcium sulfate (dehydrate) having a final concentration of 2 mmol/L and 0.585 g of sodium chloride having a final concentration of 10 mmol/L and dissolved, and further 2 mL of a 1 mol/L acetate buffer solution having a final concentration of 2 mmol/L, 0.5 mL of a 10% Triton-X 100 solution having a final concentration of 0.005%, and water were added to the solution so that the total amount of the solution was 1,000 mL, to thereby obtain a diluent.

—Preparation 2 (Preparation of Borax Buffer Solution)—

In a vessel, 90 mL of water was added to 19.07 g of 4 sodium borax (decahydrate) and 9.0 g of sodium chloride and dissolved. The obtained solution was adjusted with concentrated hydrochloric acid so as to have a pH of 8.5, and further water was added to the solution and adjusted so that the total amount of the solution was 1,000 mL, to thereby obtain a 0.05 mol/L borax buffer solution.

—Preparation 3 (Preparation of Fibrinogen Solution)—

In a conical flask, lOmL of a 0.05 mol/L borax buffer solution was poured, 96 mg of a fibrinogen (Fibrinogen Fraction I Type I-S, manufactured by Sigma Chemical Co., bovine blood plasma-derived, PRODUCT NUMBER F8630) was added thereto, left as it was and dissolved. Then, the solution was filtered to remove the suspended matter to thereby obtain a 0.72% fibrinogen.

—Preparation 4 (Preparation of Thrombin Solution)—

In a vessel, a thrombin (manufactured by Sigma Chemical Co., bovine blood plasma-derived, PRODUCT NUMBER T6634) was dissolved in a 0.05 mol/L borax buffer solution to prepare a thrombin solution having a concentration of 1,000 U/mL. The thrombin solution having a 1,000 U/mL was diluted 50-fold using a 0.05 mol/L borax buffer solution, and the diluted solution was used in the test for measuring the activity of natto-kinase.

### Example 12

An agent containing Bacillus natto culture extract was prepared in the same manner as Example 11 except that the added amount of the maltose was changed to 20% by mass. The activity of natto-kinase of the agent was measured. Table 3 shows the resultant values.

### Example 13

An agent containing Bacillus natto culture extract was prepared in the same manner as Example 11 except that the added amount of the maltose was changed to 40% by mass. The activity of natto-kinase of the agent was measured. Table 3 shows the resultant values.

### Example 14

An agent containing Bacillus natto culture extract was prepared in the same manner as Example 13 except that a reduced dextrin (H-PDX, manufactured by Matsutani Chemical Industry Co., Ltd.) was used instead of the maltose. The activity of natto-kinase of the agent was measured. Table 3 shows the resultant values.

### Example 15

An agent containing Bacillus natto culture extract was prepared in the same manner as Example 13 except that a fructose was used instead of the maltose. The activity of natto-kinase of the agent was measured. Table 3 shows the resultant values.

### Example 16

An agent containing Bacillus natto culture extract was prepared in the same manner as Example 13 except that a sorbitol was used instead of the maltose. The activity of natto-kinase of the agent was measured. Table 3 shows the resultant values.

### Comparative Example 5

An agent containing Bacillus natto culture extract was prepared in the same manner as Example 11 except that the maltose was not added to the Bacillus natto culture extract. The activity of natto-kinase of the agent was measured. Table 3 shows the resultant values.

TABLE 3

| | | | | | | | | Compara. |
|---|---|---|---|---|---|---|---|---|
| | | Ex. 11 | Ex. 12 | Ex. 13 | Ex. 14 | Ex. 15 | Ex. 16 | Ex. 5 |
| Saccharides | Maltose | 10 | 20 | 40 | — | — | — | — |
| | (% by mass) | | | | | | | |
| | Reduced Dextrin | — | — | — | 40 | — | | — |
| | (% by mass) | | | | | | | |
| | Fructose | — | — | — | — | 40 | — | — |
| | (% by mass) | | | | | | | |
| | Sorbitol | — | — | — | — | — | 40 | — |
| | (% by mass) | | | | | | | |
| Activity of | Start of | 2,290 | 2,330 | 2,280 | 2,190 | 2,270 | 2,050 | 2,310 |
| Natto-kinase | Preservation | | | | | | | |
| (FU) | 6 months later | 2,305 | 2,310 | 2,180 | 2,200 | 2,000 | 1,910 | 2,200 |
| | 12 months later | 2,320 | 2,270 | 2,340 | 2,150 | 1,750 | 1,840 | 1,700 |
| | 24 months later | 2,150 | 2,050 | 2,200 | 2,180 | 1,760 | 1,790 | 1,250 |

The above results exemplified that an agent containing Bacillus natto culture extract which includes a Bacillus natto culture extract and any one of a reducing sugar and a reducing sugar derivative is capable of preventing degradation of natto-kinase over long periods, preventing itself from becoming darker in color with the lapse of time, as well as preventing the occurrence of natto-specific odor. Accordingly, it is possible for even people who do not like natto to take the agent containing the Bacillus natto culture extract.

What is claimed is:

1. An agent containing Bacillus natto culture extract comprising:
a Bacillus natto culture extract, and
any one of a reducing sugar and a reducing sugar derivative.

2. The agent containing Bacillus natto culture extract according to claim 1, wherein the Bacillus natto culture extract comprises natto-kinase.

3. The agent containing Bacillus natto culture extract according to claim 1, wherein the reducing sugar is a maltose.

4. The agent containing Bacillus natto culture extract according to claim 1, wherein the reducing sugar derivative is a reduced dextrin.

5. The agent containing Bacillus natto culture extract according to claim 1, wherein the added amount of the reducing sugar is 50 parts by mass to 150 parts by mass relative to 100 parts by mass of solids of the Bacillus natto culture extract.

6. The agent containing Bacillus natto culture extract according to claim 1, wherein the added amount of the reducing sugar derivative is 50 parts by mass to 150 parts by mass relative to 100 parts by mass of solids of the Bacillus natto culture extract.

7. The agent containing Bacillus natto culture extract according to claim 1, being a capsulated agent.

8. The agent containing Bacillus natto culture extract according to claim 1, being a tablet agent.

9. The agent containing Bacillus natto culture extract according to claim 1, being a liquid agent.

10. A preservation method of Bacillus natto culture extract comprising:
preserving a Bacillus natto culture extract such that the Bacillus natto culture extract and any one of a reducing sugar and a reducing sugar derivative are mixed.

11. The preservation method of Bacillus natto culture extract according to claim 10, the Bacillus natto culture extract contains natto-kinase.

12. The preservation method of Bacillus natto culture extract according to claim 10, the reducing sugar is a maltose.

13. The preservation method of Bacillus natto culture extract according to claim 10, the reducing sugar derivative is a reduced dextrin.

14. The preservation method of Bacillus natto culture extract according to claim 10, the added amount of the reducing sugar is 50 parts by mass to 150 parts by mass relative to 100 parts by mass of solids of the Bacillus natto culture extract.

15. The preservation method of Bacillus natto culture extract according to claim 10, the added amount of the reducing sugar derivative is 50 parts by mass to 150 parts by mass relative to 100 parts by mass of solids of the Bacillus natto culture extract.

16. The preservation method of Bacillus natto culture extract according to claim 10, the Bacillus natto culture extract and any one of the reducing sugar and the reducing sugar derivative are mixed and preserved in a hermetically-sealed container.
